(19) United States
(12) Patent Application Publication
(10) Pub. No.: US 2014/0088380 A1
(43) Pub. Date: Mar. 27, 2014

(54) Systems and Methods for Real Time Multispectral Imaging

(71) Applicants: Stephen H. Sprigle, Marietta, GA (US); Linghua Kong, Marietta, GA (US); Jayme J. Caspall, Decatur, GA (US); Mark G. Duckworth, Acworth, GA (US)

(72) Inventors: Stephen H. Sprigle, Marietta, GA (US); Linghua Kong, Marietta, GA (US); Jayme J. Caspall, Decatur, GA (US); Mark G. Duckworth, Acworth, GA (US)

(73) Assignee: Georgia Tech Research Corporation, Atlanta, GA (US)

(21) Appl. No.: 13/939,134

(22) Filed: Jul. 10, 2013

Related U.S. Application Data
(62) Division of application No. 12/518,824, filed on Feb. 5, 2010, now abandoned.
(60) Provisional application No. 60/869,822, filed on Dec. 13, 2006.

(51) Int. Cl. A61B 5/00

(52) U.S. Cl. CPC A61B 5/445; A61B 5/0077
USPC 600/306

(57) ABSTRACT

Multispectral filter arrays and methods of making and using the arrays are described herein. Multispectral imaging systems and methods of making and using the systems are also described. A multispectral filter array includes a mosaic of light sensitive elements that includes a first element sensitive to a first narrow spectral region, a second element sensitive to a second narrow spectral region, and a third element sensitive to a third narrow spectral region. A multispectral imaging system includes an illumination system having at least three lighting elements, each of which are configured to transmit light at a different wavelength, and the multispectral filter array.

### RELATED APPLICATIONS

This application is a divisional of U.S. patent application Ser. No. 12/518,824, filed on Feb. 5, 2010, which published as U.S. Patent Publication No. 2010/0140461, on Jun. 10, 2010, and which is the National Phase of International Application No. PCT/US07/87479, filed on Dec. 13, 2007, which published in English as WO 2008/074019, on Jun. 19, 2008, and which claims benefit to U.S. Provisional Application No. 60/869,822 filed on Dec. 13, 2006. The disclosures of these documents are hereby incorporated by reference in their entirety as if fully set forth below.

### GOVERNMENT LICENSE RIGHTS

This invention was made with U.S. Government support under Grant No. 1 RO1 EB002224-01 awarded by the National Institutes of Health. The U.S. Government has certain rights in the invention.

### TECHNICAL FIELD

The present invention is directed generally to the field of multispectral imaging. More specifically, the present invention is directed to systems and methods for real time multispectral imaging of surfaces and subsurfaces.

### BACKGROUND OF THE INVENTION

Detection, identification, and characterization of erythema and bruising are important to the prevention and diagnosis of pressure ulcers as well as the assessment and prevention of instances of abuse, respectively.

Erythema is an abnormal redness or inflammation of a mucosal or dermal surface caused by dilation of superficial capillaries. Erythema can result from many different causes, including but not limited to, diseases of the mucosa and skin, systematic diseases, infection of the mucosa and skin, and physical insults to biological tissues, such as pressure-induced ischemia. In more severe forms, erythema can cover large areas of the body and can include a component of ulceration, either solitary or widespread. Localized erythema is a component of a post-ischemic response, which can signal reactive hyperemia or the inflammation response associated with a Stage I pressure ulcer. If left undetected, pressure-induced ischemia can result in tissue damage or necrosis, manifested as a Stage III or Stage IV pressure ulcer.

Pressure ulcers continue to be a serious secondary complication for people with impaired mobility and sensation and as such have been identified as a public health concern. The Agency for Health Care Policy and Research estimated that one (1) to three (3) million adults experience pressure ulcers, resulting in an average cost range of $500 to $40,000 to treat and heal each ulcer. Annual Medicare spending is conservatively approximated at $1.34 billion for the treatment of pressure ulcers. Not only are pressure ulcers monetarily costly, but they have also been associated with increased mortality and morbidity.

Early identification of post-ischemic erythema is clinically very important in the treatment of pressure ulcers, considering that early intervention can prevent progression into more serious Stage III or Stage IV pressure ulcers. Currently, clinicians visually assess the skin to identify the existence and extent of erythema. However, skin pigmentation (due to the presence of melanin) can mask the indicators of erythema visible to the unaided eye, thereby hindering clinical assessment of pressure ulcers in people with darkly pigmented skin. Evidence suggests that Stage I pressure ulcers in darkly pigmented patients are more likely to go undetected and to deteriorate into Stage III or Stage IV pressure ulcers as compared to the prognosis for lightly pigmented patients.

Similar to the pathophysiology of erythema, a bruise is result of a physical insult to the skin that causes capillary damage, permitting blood to seep into the tissue subject to and adjacent to the site of insult. Bruises often induce pain but are not by themselves normally dangerous. However, bruises can be indicative of serious, life-threatening injuries, such as hematomas, fractures, and internal bleeding. Further, bruising provides a window into life-threatening situations as bruising is the earliest and most visible sign of abuse.

Statistics from the U.S. Department of Health and Human Services indicate that 2.9 million suspected cases of possible child abuse were reported to child protective service agencies, of which an estimated 906,000 of these reports were substantiated. It is estimated that more than four (4) children die per day as a result of child abuse in the home. Incidents of elder abuse are rapidly approaching the prevalence of child abuse, as an estimated one (1) to two (2) million elderly Americans have been injured, exploited, or otherwise mistreated by someone on whom they depended for care or protection.

Detection and documentation of bruising is an effective means for the assessment and prevention of abuse. Visual inspection of intact skin in vivo and photography of bruises are two conventional methods for clinically assessing bruising. Analysis of bruises, particularly determining the age of bruises based on visual appearance alone is qualitative, subjective, inaccurate, and hence unreliable. The unreliability of these methods is further accentuated by the presence of melanin in the skin.

Current methods known in the art for the detection and characterization of erythema and bruising are highly subjective, not reproducible, and often do not detect disease at an early stage when treatment and preventive strategies are most effective. In addition, these methods are dependent upon patient cooperation, patient skin pigmentation, examiner direct line of sight, and available lighting.

Consequently, there is a need of for an improved, non-invasive means for detection, identification, and characterization of erythema and bruising beyond visual inspection with the unaided eye. Recently, spectroscopy has emerged as a technology that could be utilized to improve the reliability of erythema and bruise detection. Spectroscopy resolves the phenomenon of the interaction of light and matter by analyzing the dispersion of a target object's light into its component colors. By performing this dissection and analysis of a target object's light, one can infer the physical properties of that object, such as its composition. For example, when skin is illuminated by light, the light can be redirected by reflection, scattering, or fluorescence. It is known in the art that the interaction of light at its different constituent wavelengths with a target can provide different information about that target. In addition, as the wavelength of the light decreases, its depth of penetration into the skin also increases. Chromophores located at different depths in the tissue therefore absorb, scatter, reflect and re-emit light at various wavelengths.

Tissue Reflectance Spectroscopy (TRS) provides the fractional contents of various chromophores by measuring the optical reflectivity of one sample point at a time for a continuous range of wavelengths at fine steps (e.g. two (2) nm incremental steps). Although TRS is a reliable tool to investigate the basic biochemical process associated with an injured biological surface on both lightly and darkly pigmented skin, point spectroscopy is too arduous a process to perform in a clinical setting. Specifically, TRS is performed at one spatial point at a time. Therefore, creation of a spatial distribution map of the chromophore concentration over time using point spectroscopy is time consuming, tedious, and subject to risk of location error and movement error. Such a distribution map, however, is important to erythema and bruise detection and characterization as it contains the intrinsic features of the diseased skin, such as its shape, size, and age.

An alternative spectroscopy approach to TRS is multispectral imaging. The difference between the two technologies is that TRS samples more densely in the spectral domain and more sparsely over the spatial domain than multispectral imaging. Therefore, multispectral imaging permits the convenient collection of images of millions of sample surfaces at a set of discrete wavelengths using band pass filters to remove unimportant spectra. Consequently, multispectral imaging has matured into a technology with many applications, including classification of targets in defense, produce sorting, precision farming in agriculture, product quality online inspection in manufacturing, contamination detection in the food industry, remote sensing in mining, atmospheric composition monitoring in environmental engineering, and early stage diagnosis of cancer and tumors. Implementation of multispectral imaging technology to date has required contact between the transducer and surface of interest; and/or cumbersome, non-portable equipment, including numerous cameras and associated filters; and/or fusion of multiple images taken at different wavelengths to create a single composite image; and/or controlled lighting conditions.

Thus, there is a need for a simple, affordable, non-contact, hand-held device that permits real time multispectral imaging of surfaces and subsurfaces with a single image under ambient light conditions, such as, but not limited to, a clinical setting. In addition, there is a need for multispectral imaging technology that is capable of detecting and characterizing erythema and bruises on surfaces and subsurfaces through isolation of spectra of interest. Further, there is a need for multispectral imaging technology that is capable of detecting and characterizing erythema and bruises independent of skin pigmentation.

### BRIEF SUMMARY OF THE INVENTION

The various embodiments of the present invention are directed to multispectral filter arrays, multispectral imaging systems, and methods of making and using the arrays and systems. Broadly described, a multispectral filter array includes a mosaic of light sensitive elements. The mosaic has a first element sensitive to a first narrow spectral region of wavelengths, a second element sensitive to a second narrow spectral region of wavelengths, and a third element sensitive to a third narrow spectral region of wavelengths. The multispectral filter array can further include a fourth element sensitive to a fourth narrow spectral region of wavelengths.

The first, second, third, and fourth light sensitive elements can be arranged in a grouping. One grouping of light sensitive elements can be arranged in alternating rows of pairs of the light sensitive elements. The mosaic can be formed of a plurality of the groupings.

The narrow spectral regions of wavelengths can cover less than or equal to about 100 nanometers. In other cases, the narrow spectral regions of wavelengths can cover less than or equal to about 10 nanometers, and even down to less than or equal to about 2 nanometers. In some situations, the narrow spectral region can be a single nanometer. Depending on the application, the smaller the narrow spectral region of wavelengths, the more sensitive the detection capability of the filter as will be described in more detail below.

By way of example, the first narrow spectral region can cover a wavelength of about 460 nm, the second narrow spectral region can cover a wavelength of about 540 nm, the third narrow spectral region can cover a wavelength of about 577 nm, and the fourth narrow spectral region can cover a wavelength of about 650 nm. Other embodiments of approximately single wavelength narrow spectral regions are described and claimed.

A multispectral imaging system for detecting a target can include the multispectral filter array above and an illumination system having at least three lighting elements. The at least three lighting elements each transmit light at a different wavelength to the target. In some embodiments, the illumination system has a fourth lighting element configured to transmit light at a different wavelength from each of the at least three lighting elements and the mosaic of light sensitive elements has a fourth element sensitive to a fourth narrow spectral region of wavelengths. Light-emitting diodes can be used as the lighting elements.

The system can generate a real-time, multispectral image of the target without contacting a surface of the target. It can also be portable and/or hand-held. The system can also include a sensor element in communication with the filter. One such sensor is a photosensor. The photosensor element can be a complimentary-symmetry metal oxide semiconductor sensor. Alternatively, it can be a charged-coupled device sensor.

The system can also include a processing unit in communication with the sensor unit. It can also include a lens in optical communication with the filter and configured to focus the at least three different wavelengths transmitted to the target. The system can also include a polarizing filter disposed in communication between the lens and the filter.

In some embodiments, the target can be erythema or a bruise. For example, the target can be a biological surface or subsurface. The biological surface or subsurface can include an erythema or a bruise that can be detected.

According to other embodiments, a method of multispectral imaging includes illuminating a target with light having at least a first, second, third, and fourth wavelength. The method also includes filtering the light with a filter array, wherein the filter array comprises a mosaic of light sensitive elements comprising a first element sensitive to a first narrow spectral region of wavelengths, a second element sensitive to a second narrow spectral region of wavelengths, and a third element sensitive to a third narrow spectral region of wavelengths, and the fourth spectral region of wavelengths. A feature of the target can be calculated from the filtered light. A multispectral image of the target feature can be displayed in real time. In some cases, the light scattered by the target can be focused.

Other aspects and features of embodiments of the present invention will become apparent to those of ordinary skill in the art, upon reviewing the following detailed description in conjunction with the accompanying figures.

### BRIEF DESCRIPTION OF DRAWINGS

The various embodiments of the invention can be better understood with reference to the following drawings. The components in the drawings are not necessarily to scale, emphasis instead being placed upon clearly illustrating the principles of the various embodiments of the present invention. In the drawings, like reference numerals designate corresponding parts throughout the several views.

FIG. 1 is a micrograph of a mosaic filter.

FIG. 2A graphically depicts an existing commercially-available RGB wide band profile generated by a Bayer color filter array.

FIG. 2B graphically depicts the narrow bands (<20 nm) at 460 nm, 540 nm, 577 nm, and 650 nm generated using multi layer films of oxygenated metal.

FIG. 2C graphically depicts the narrow bands (<20 nm) at 540 nm, 577 nm, 650 nm, and 970 nm generated using multi layer films of oxygenated metal.

FIGS. 3A-C graphically depict the absorbance curves of oxy-hemoglobin, deoxy-hemoglobin, bilirubin, water, and melanin.

FIG. 4 is a schematic of the mosaic filter.

FIG. 5 is a schematic of the LED-based illumination system

FIG. 6 graphically depicts the spectral power distribution of the LED system.

FIG. 7A schematically represent the organization of data on the sensor.

FIG. 7B schematically represents the output of reconstruction.

FIG. 7C illustrates the spectral response of a bruise at four wavelengths.

FIG. 8 graphically depicts distribution of the subjects' skin color.

FIGS. 9A-E illustrate the fusion results of five representative subjects based on the four most effective algorithms.

FIG. 10 graphically depicts skin color distribution of enhanced versus unenhanced subjects.

FIGS. 11A-D graphically depict the mean NBR versus wavelength at different ages.

FIG. 12 graphically illustrates source signals IC1, IC2 and IC3 estimated by ICA.

FIGS. 13A-C graphically depict the differential concentration and path length for the three estimated path length versus the age of the bruise.

FIGS. 14A-B graphically depict the differential concentration and path length for S-Lit₂.

FIGS. 15A-B graphically depict the differential concentration and path length for S-Lit₂.

### DETAILED DESCRIPTION OF PREFERRED EMBODIMENTS

The various embodiments of the present invention are directed to multispectral filter arrays, and methods of making and using the arrays. Other embodiments are directed to multispectral imaging systems and methods of making and using the systems. The multispectral imaging systems make use of the multispectral filter array.

The multispectral filter arrays generally include a one or more mosaics of light sensitive elements. Each mosaic has a first element sensitive to a first narrow spectral region of wavelengths, a second element sensitive to a second narrow spectral region of wavelengths, and a third element sensitive to a third narrow spectral region of wavelengths. In exemplary embodiments, each mosaic has a fourth element sensitive to a fourth narrow spectral region of wavelengths.

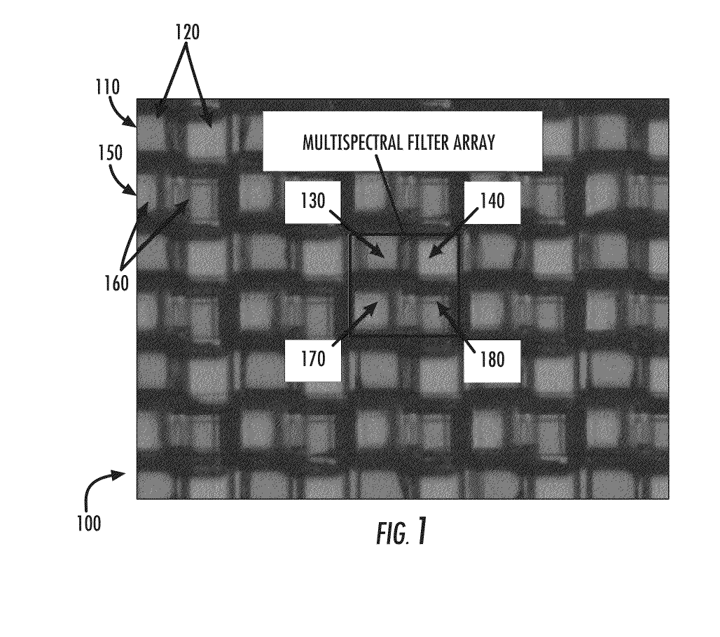

An embodiment of the present invention is a filter that targets at least four (4) wavelengths. In one embodiment, the filter comprises mosaics of selectively transmissive filters superimposed disposed on an imaging array. In one embodiment, which is illustrated in FIG. 1, the mosaic 100 comprises repeating binary rows of alternating filter tiles, wherein a first row 110 is comprised of a first repeating pair of tiles 120, with the first member of the first pair 130 having a first narrow band transmission profile and the second member of the first pair 140 having a second narrow band transmission profile, and a second row 150 comprised of a second repeating pair of tiles 160, with the first member of the second pair 170 having a third narrow band transmission profile and the second member of the second pair 180 having a fourth narrow band transmission profile.

The narrow band profiles can encompass light having wavelength difference of approximately 100 nm (e.g. about 450 nm to about 550 nm). In one embodiment, the narrow band profile can encompass light having a wavelength difference of approximately 25 nm. In an exemplary embodiment, the narrow band profile would be less than approximately 5 nm. Embodiments where the narrowness of light is less than 5 nm increases the specificity of the filter, but decreases the sensitivity of the filter. In application where greater specificity is required, a narrow band profile would be desired, which could encompass light having a wavelength difference of approximately 1 nm.

In an embodiment, the mosaic filter can be utilized with complimentary-symmetry metal oxide semiconductor (CMOS) technology, among others. CMOS technology offers adequate performance at a low cost and can be easily integrated with board-level electronics. In a CMOS sensor, each pixel has its own charge-to-voltage conversion, and the sensor can include amplifiers, noise-correction, and digitization circuits, so that the chip outputs digital bits. These other functions, however, increase the design complexity and reduce the area available for light capture. Given that each pixel performs its own conversion, uniformity is reduced relative to similar charge-coupled device (CCD) technology, but the chip can be constructed to require less off-chip circuitry for basic operation, and a CMOS sensor provides low power dissipation and the possibility of a smaller system size.

In another embodiment, the mosaic filter can be utilized with charge-coupled device (CCD) based technology, among others. In a CCD sensor, every pixel's charge is transferred through a very limited number of output nodes, which will be converted to voltage, buffered, and sent off-chip as an analog signal. All of the pixels in a CCD sensor can be devoted to light capture, and the output's uniformity is high. CCD sensors have traditionally provided the performance benchmarks in the photographic, scientific, and industrial applications that demand the highest image quality at the expense of system size and power consumption.

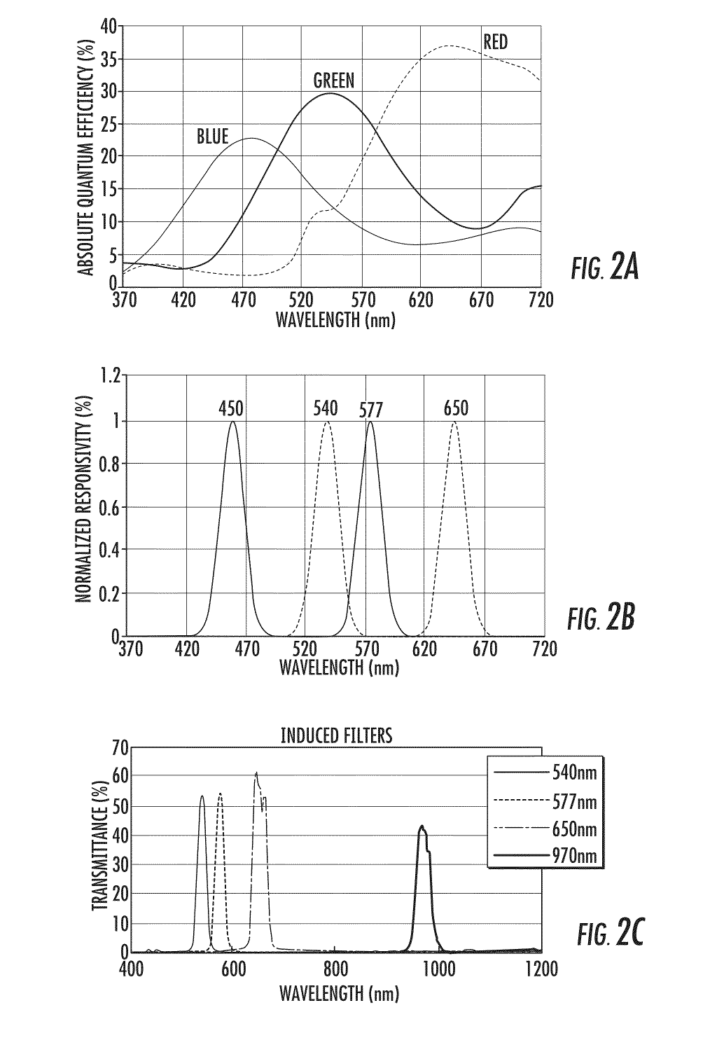

Color digital cameras utilize a color filter array (mosaic), such as the Bayer mosaic, which consists of tiled red, green, and blue (RGB) filters that sit atop a photosensitive sensor array, typically a CMOS or CCD array. As illustrated in FIG. 2A, each of the tiles in the Bayer mosaic produces a wide band transmission profile. The cameras reconstruct images from three (3) filters, but the smallest square unit cell of the mosaic is comprised of four (4) filter tiles, having two green elements, one red, and one blue. Images are reconstructed from the Bayer mosaic filter arrangement using many existing demosaicing algorithms, which may be implemented in real time in the camera firmware or in post processing from the raw digitized mosaic response. Generally, each resulting pixel of the demosaiced image is a linear superposition of some subset of pixels in its local vicinity. Consequently, each filter in the unit cell contributes to the colorization of every pixel in the unit cell.

In the standard case of digital photography, the objective of demosaicing is to faithfully reproduce the colorization in the original target. Various embodiments are conceived upon an analogy with the digital color camera, but differ significantly via its intention to spectrally enhance specific features of the surface. The filters are not intended to faithfully capture the target, but instead to capture the target with an exaggerated bias toward known spectral features. In fact, the spectral band transmitted by any tile in the unit cell of the mosaic need not fall within the visible range. To the extent that specific filter bands define a subspace of the spectral domain in which the targeted feature may be well-characterized to stand out against other features that may be present within a target, a filter mosaic can be conceived such that real time demosaicing will generate a real time contrast-enhanced image.

In one embodiment of the present invention, a mosaic filter fabricated to include at least four (4) types of tiles (FIG. 1), wherein each type of tile of the unit cell provides a unique narrow band transmission profile, as demonstrated in FIGS. 2B-C. The filters in the mosaic are chosen according to the requirements of the particular application such that an identified demosaicing algorithm may be applied to provide the desired contrast enhancement or noise removal for the targeted feature identification.

The mosaic filter array can be fabricated on any substrate, provided that the substrate does not interfere substantially or materially affect performance of the filter array. In one embodiment, the mosaic filter can be fabricated on a glass substrate or the like. The glass substrate can be approximately 0.3 mm to approximately 0.5 mm thick. The mosaic filter disposed on the substrate can then be laminated on the surface of a photosensor, such as a CMOS sensor or CCD sensor, among others. In another embodiment, the mosaic filter can be disposed on a layer of glass on the surface of a photosensor, such as a CMOS sensor or CCD sensor, among others. The layer of glass can be approximately 0.1 mm thick.

In an embodiment, the mosaic filter can be fabricated as a “checkerboard” filter array, as demonstrated in FIG. 1. Precisely-shaped masks can be used for fabricating filter film structures at a designated position on the substrate. The masks can be fabricated by optical lithography technology, which is known in the art of semiconductor micro-manufacturing. In order to fabricate filter mosaic, at least four (4) kinds of masks are needed, which can both prevent the transmittance of ultraviolet (UV) light and can project a selected geometry onto a substrate. First, a photoresist compound is applied to coat a clean substrate, (e.g., glass). Following solidification of the photoresist, the first mask is applied to the substrate. The masked substrate is then exposed to an UV light environment. After exposure of the masked substrate to UV light, the portions of the photoresist that were exposed to UV light are removed by liquid erosion to reveal the underlying substrate. The remaining photoresist that was unexposed to UV light forms the first mask for deposition. This optical lithography process is repeated until the at least four kinds of masks are fabricated onto the substrate.

Upon completion of the optical lithography process, deposition of multi-layer thin films can be performed by the process of physical vapor deposition (PVD) within a vacuum chamber. Multi-layer thin films can be constructed by sandwiching at least three (3) kinds of coating materials, including but not limited to TiO₂, Al₂O₃, and Ag to form an optical thin film system comprising approximately twelve (12) layers having a thicknesses ranging from approximately 40 nm to approximately 150 nm. The coating materials of TiO₂, AL₂O₃, and Ag can be heated by an electron beam gun to generate a vapor of the coating material within a vacuum chamber. The pressure in the vacuum chamber ranges from approximately 0.01 Pascals (Pa) to approximately 0.02 Pa. The vapor of a coating material can then coagulate on the surface of the substrate to form a thin film. The substrate undergoing PVD is periodically bombarded by an argon (Ar) ion beam, which serves a dual function: removal of potential molecular layers of water that can accumulate on the substrate, and to activate the surface of the substrate, improving adhesion of the coating material to the substrate. In order to improve the environmental characteristics of the coatings, Ar ion bombardment can be conducted throughout the deposition process to increase the density of the coating micro-configurations. The thickness of each thin film layer system can be monitored using a monochrome photometer. The accuracy of the monitoring of thin film thickness is determinative of the spectral properties of each thin film.

The conditions of the PVD process can be varied depending upon the coating material used, the composition of residual gasses within the deposition chamber, including but not limited to H₂O, N₂, O₂, H₂, and oil vapor, among others, and the substrate temperature (approximately 80° C.). The deposition rate, ranging from approximately 0.3 nm/s to approximately 2 nm/s, can also affect the performance of the coatings. The PVD process is repeated for each of the at least four (4) filters comprised of at least four (4) coatings to fabricate a mosaic filter.

The mosaic filter can be used to interrogate the multispectral reflectivity or re-emission of many surfaces. The mosaic filter can be used in a variety of applications, including but not limited to health-related diagnosis techniques, produce sorting, classification of targets in defense, precision farming in agriculture, product quality online inspection in manufacturing, contamination detection in the food industry, remote sensing in mining, and atmospheric composition monitoring in environmental engineering.

A system for the detection, identification, and characterization of surfaces and subsurfaces can include a mosaic filter, an illumination system, a detection means, an imaging processing system, and a user interface, wherein the mosaic filter narrowly targets at least four defined wavelengths. In one embodiment, this system detects erythema and/or bruises.

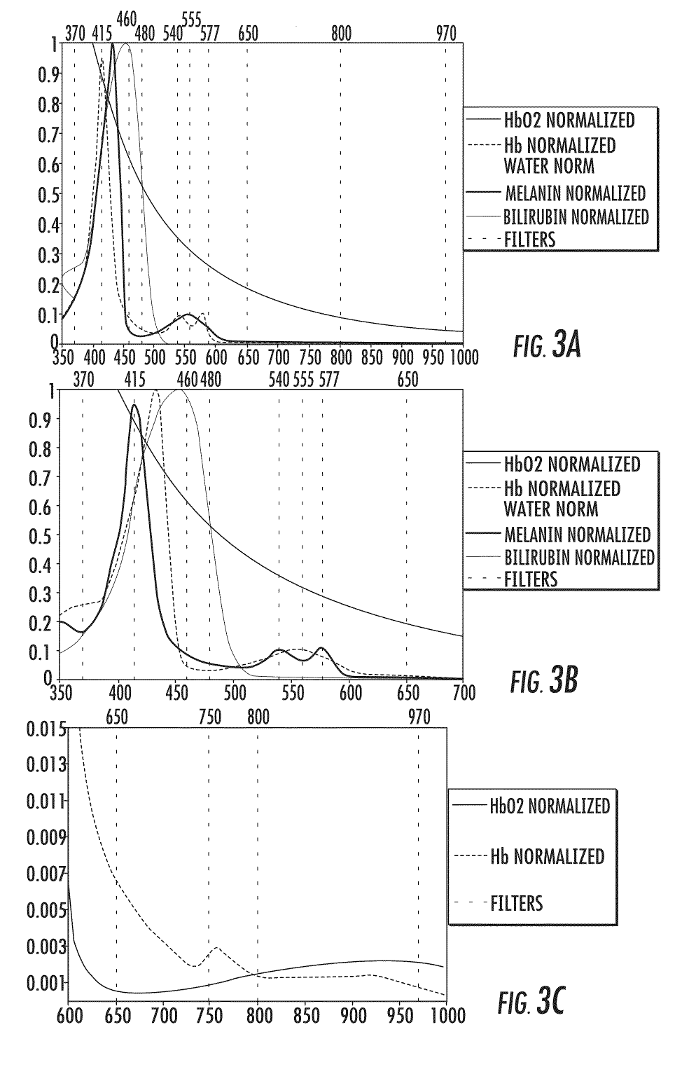

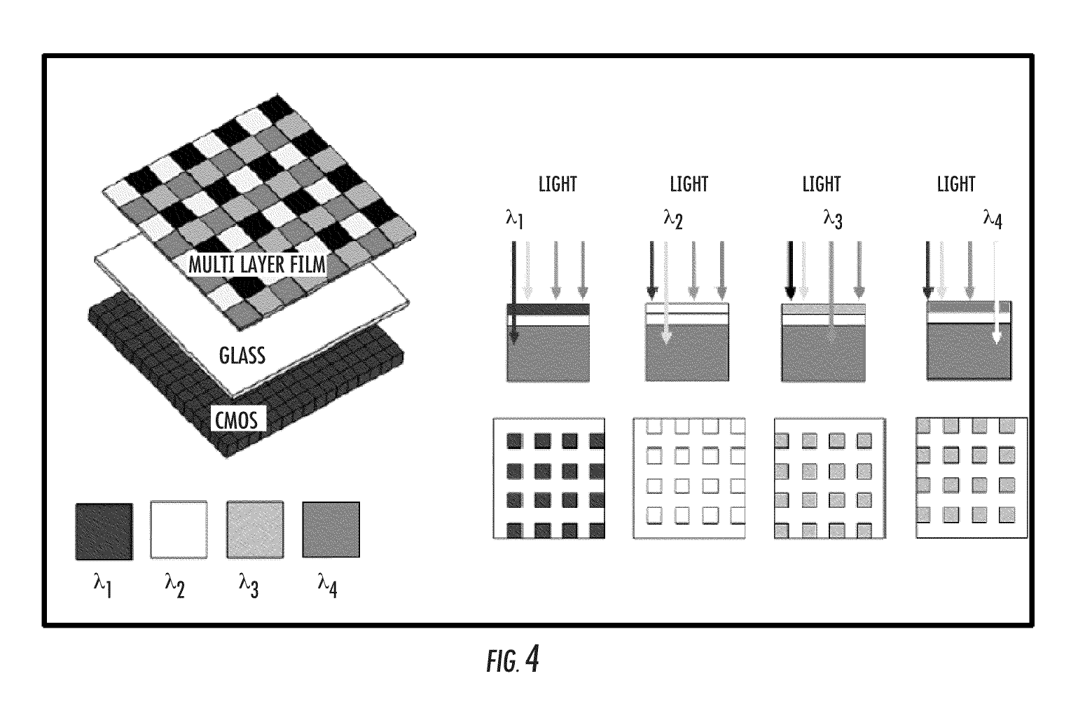

Biologically, the primary chromophores of interest when detecting erythema are oxy-hemoglobin (oxyHb), deoxy-hemoglobin (Hb), and melanin. FIGS. 3A-C illustrates that Hb and oxyHb absorb light in the same spectral range but with different absorption peaks. The absorption of melanin decreases over the spectral range from approximately 400 nm to approximately 1100 nm (FIGS. 3A-C). For individuals with darkly pigmented skin, the absorption by melanin is typically greater than that of hemoglobin in the visible spectra of approximately 500 nm to approximately 600 nm. This masking by melanin hinders erythema detection. Multispectral imaging with a mosaic filter will filter out unwanted spectra and permit detection of erythema.

In one embodiment, multispectral erythema and bruise analysis can be performed using a mosaic filter that narrowly targets four (4) defined wavelengths of approximately 460 nm, approximately 540 nm, approximately 577 nm, and approximately 650 nm (FIG. 2B). In this embodiment, the 460 nm filter targets bilirubin, the 540 nm filter targets oxyHb, and Hb, the 577 nm filter targets oxyHb, and the 650 nm filter targets melanin and provides a background image. This filter array facilitates both the analysis of the presence of bilirubin over time, and can be used for erythema analysis (i.e., by using the 577 nm filter and the 650 nm filter). Specifically, this filter at 540 nm takes advantage of the isobestic points of oxyHb and Hb in the UV and visible spectral ranges. It permits desensitized subtraction of hemoglobin from the 460 nm filter, enhancing the detection of bilirubin.

In another embodiment, multispectral erythema analysis can be performed using a mosaic filter that narrowly targets four defined wavelengths of approximately 540 nm, approximately 577 nm, approximately 650 nm, and approximately 970 nm. The spectral transmission of this embodiment of the mosaic filter is shown in FIG. 2C. Both the 540 nm filter and the 577 nm filter provide estimates of oxyHb. The 650 nm filter targets melanin and provides a background image. The 970 nm filter targets the water peak and permits deep penetration of the surface.

In another embodiment, multispectral erythema and bruise analysis can be performed using a mosaic filter that narrowly targets four defined wavelengths of approximately 460 nm, approximately 560 nm, approximately 577 nm, and approximately 650 nm. In this embodiment, the 460 nm filter targets bilirubin, the 560 nm filter targets Hb, the 577 nm filter targets oxyHb, and the 650 nm filter targets melanin and provides a background image. This filter array targets facilitates bilirubin, Hb, and oxyHB at points where their reflectance are relatively high, and can be used for erythema analysis (i.e., by using 560 nm, 577 nm, and 650 nm filters).

In yet another embodiment, multispectral bruise analysis can be performed using a mosaic filter that narrowly targets four defined wavelengths of approximately 460 nm, approximately 525 nm, approximately 560 nm, and approximately 577 nm. The 460 nm filter targets bilirubin, whereas the 525 nm filter targets melanin, bilirubin, Hb, and oxyHb. The 560 nm filter provides an estimate of Hb, and the 577 nm filter targets oxyHb. This filter permits elucidation of the ration of Hb and oxyHb.

In still another embodiment, multispectral erythema analysis can be performed using a mosaic filter that narrowly targets four defined wavelengths of approximately 505 nm, approximately 545 nm, approximately 568 nm, and approximately 615 nm. Both the 505 nm filter and the 615 nm filter allow for analysis of the melanin curve. In contrast, the 545 nm filter and the 568 nm filters provide estimates of oxyHb and Hb at their isobestic points.

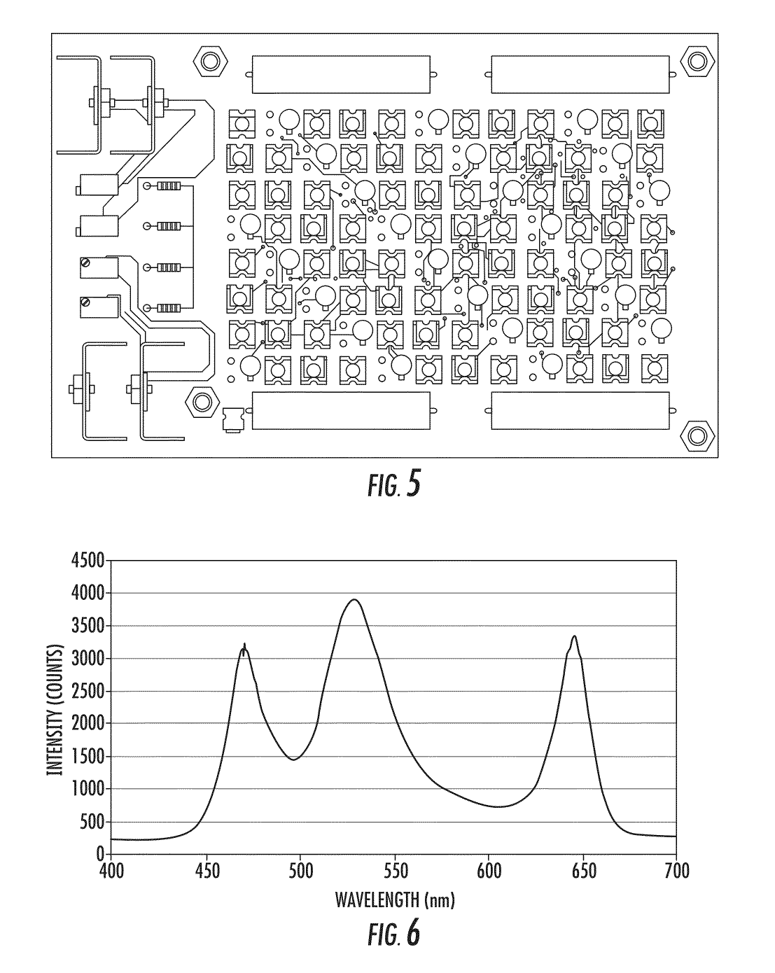

In one embodiment, the mosaic filter can be fabricated and applied to a CMOS sensor in an analogous manner to traditional design. The crafting process can generate a multilayer film filter on a glass substrate using a vacuum ion beam splitter and lithographic techniques, as described above. This approach is illustrated in FIG. 4 and results in a narrow band spectral response synched to at least four (4) defined wavelengths (FIG. 2B).

High image resolution is desired for distinguishing fine spatial variations of the chromophore concentrations in the tissue under interrogation. Narrow band filters are often desired for effective feature extraction from a multispectral mosaic response. Due primarily to the large number of deposited layers, it is currently not possible to fabricate narrow band filters with lateral dimensions on the scale of the actual pixel pitch of currently available sensor arrays (<10 um). In such practical, narrow band, implementations, each tile within the mosaic unit cell is comprised of several sensor array pixels. This is only a minor practical limitation. While reducing the resolution below the sensor array physical limit, it has little effect on the basic operation of the system. In one particular approach, but not limited to, the minimum area of interest for consideration of bruising on the skin surface is defined as one (1) mm², which requires the need to “sample” at 0.5 mm. For a target size of ten (10) cm×ten (10) cm and a required resolution of 0.5 mm, an element will use 200×200 pixels or a total of 160,000 pixels for a four (4) tile mosaic. The fabrication of a filter array based upon approximately 20.8 μm square filters is feasible given the fabrication technique utilized. In this case, less than one million pixels at 10 μm pitch are required to satisfy the resolution requirements. Such sensor arrays are readily available. Ultimately, however, as the technology improves, or in less demanding (larger bandwidth filters) applications, the resolution is limited by the pixel pitch in the underlying sensor array.

In one embodiment, the selection of the CMOS sensor directly impacts many other performance features of a system for erythema and bruise imaging. The CMOS technology provides near real time contrast enhancement of the hemoglobin and bilirubin content of skin In this embodiment, design criteria focused on two features: resolution and quantum efficiency. Based upon the requirements of the mosaic filter described above, the resolution and pixel size requirements of the CMOS sensor can be defined. A CMOS sensor with a resolution of 1280×1024 and pixel size of 5.2 μm meets this design requirement by providing 320×360 pixels per wavelength. In this embodiment, the 20.8 μm filter fits over four (4) CMOS pixels. In an embodiment, a filter mosaic can be designed to be 50 mm×50 mm×0.55 mm.

In the present embodiment, the block of filters for a single pixel is inversely proportional to the size of the output image. In the embodiment of a two (2)×two (2) block (FIG. 1), an output image that is ¼th the size of the filter (½ the resolution in x and y directions) is produced. If the number of wavelengths on the filter is increased above 4, thereby requiring increased block size, then the resolution of the output image will be reduced.

In one embodiment, a CMOS sensor can have a quantum efficiency over a range of approximately 460 nm to approximately 650 nm to adequately collect spectral information at the 4 wavelengths of interest. The EMPHIS300 from Photonfocus has an acceptable response. Monochrome CMOS sensors of this type are commercially available from several companies, including but not limited to Photonfocus, Eastman Kodak, Micron, Dalsa, and FillFactory.

In another embodiment, the mosaic filter can be placed on a CMOS sensor within a camera. In this embodiment, many types of monochrome CMOS sensors can be used. In one embodiment, the camera is a small unit offering 1.0 V/lux-sec sensitivity, a 60 dB dynamic range, a max 12-frame buffer, and USB bus operation.

As a multispectral imager, an embodiment of the present invention can be fitted with optics. In an embodiment, a C-mount achromatic lens from Scheiderkreunach is used, as it offers a spectral range of approximately 400 nm to approximately 1000 nm, which covers the spectrum of interest. In addition, a polarizing filter can be incorporated in an embodiment of the present invention. For those surfaces through which some penetration into the subsurface is attained, polarizing filters improve tissue image quality, reducing the specular reflection from the outer surface and more effectively capturing spectral information from the subsurface material. For example, an embodiment of the present invention can utilize Edmund's Linear Polarizing Laminated Film, among others.

An embodiment of the present invention can implement an illumination system. Generation of diffuse and uniform lighting is a difficult task given the spectral requirements and other requirements such as portability, compact size, low weight, and low power consumption. In one embodiment, a light-emitting diode (LED) based illumination can be used. As a light source, LEDs have the advantages of: (1) higher energy efficiency (approximately 50 lpw), (2) enhanced design capacity, (3) longer life (approximately 100,000 hrs), (4) lower light source temperature, (5) lower cost (approximately $0.50/per unit), and (6) fine power spectrum. The band-limited, high photonic spectral density typical of LEDs permits the light source to be tuned for the application of interest, closely matching the wavelengths of the light source with the wavelengths of the filters on the sensor array. In one embodiment, the combination of white and red LEDs results in a spectral power distribution that provides light at four (4) wavelengths of interest. The LEDs can be arranged as lattice cluster and encased with a defined geometric shape. The spectral light from different LEDs can be diffused to create a uniform and averaged light field using three layers (approximately 1.6 mm) of Soda Lime Diffusers. The thickness of the resulting case is only approximately 20 mm.

In an embodiment, the illumination system comprises at least four (4) types of LEDs synchronized to the wavelengths of the custom filter mosaic. By way of example, one such illumination system can include four (4) types of LEDs, including a SuperRed LED (624 nm, 350-450 mcd @ 70 mA, 50 deg), a White Piranha LED (1400-2000 mcd @ 50 mA, 120 deg), PureGreen Piranha LED (525 nm, 1500-2000 mcd @ 50 mA, 120 deg) and an Infrared LED (940 nm). FIG. 5 illustrates such an LED-based illumination system. FIG. 6 graphically depicts the spectral power distribution of LED-based illumination system of FIG. 5. Synchronization of the illumination system to the wavelengths detected by the array reduces the sensitivity of the detection system to ambient light.

Each LED is powered by a constant voltage supply circuit, which permits independent adjustment of the intensity of each of these four colors in order to compensate for differences in radiated power distribution. In one embodiment, the design works at a nominal voltage of 13.5 V and 1.7 A with a maximum power consumption of 23 W, a 600 lux peak illumination, and a 405 lux as average at a distance of 45 cm. In another embodiment, the design can operate at 5V and 5 W, which provides enough power for a handheld detector.

In one embodiment, the image processing system can be incorporated into the unit comprising the mosaic filter, an illumination means and detection means. In another embodiment, a laptop-based image processing system can be used.

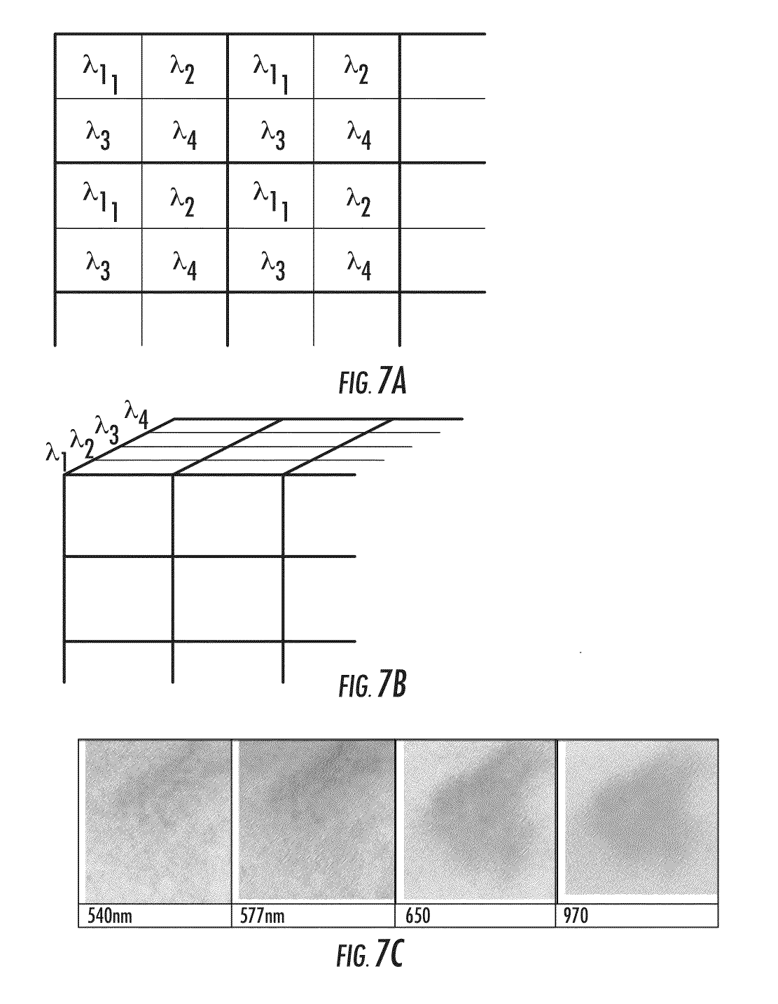

FIG. 7A represents how the data can be organized on the sensor. This illustration is also representative of the layout of the filter. The output of “reconstruction” is organized as illustrated in FIG. 7B. This organization can be referred to as an image cube, a multispectral image cube, and multi-channel image, among others. In one embodiment, the use of the mosaic filter facilitates construction of an image cube from the data collected by the sensor. Construction of the image cube can be performed by iterating over rows and columns of the sensor data and reorganizing the groups of four filters that make up a single pixel. This reconstruction is similar to that performed when using a Bayer filter array, and can be done in either the hardware or software. In an embodiment, a registration step is not required to reconstruct the image. FIG. 7C illustrates the spectral response of a bruise at four wavelengths. Further, consideration of the transmissibility of the mosaic filter coupled with the quantum efficiency of the sensor allows for normalization or a “flattening out” of the response of the sensor, such a CMOS sensor, among others.

It must be noted that, as used in this specification and the appended claims, the singular forms “a”, “an”, and “the” include plural referents unless the context clearly dictates otherwise.

All patents, patent applications and references included herein are specifically incorporated by reference in their entireties.

It should be understood, of course, that the foregoing relates only to exemplary embodiments of the present invention and that numerous modifications or alterations may be made therein without departing from the spirit and the scope of the invention as set forth in this disclosure.

The present invention is further illustrated by way of the examples contained herein, which are provided for clarity of understanding. The exemplary embodiments should not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims

### EXAMPLE 1

The objective of this study was to develop a technique using a fixed, discrete set of wavelengths that can detect erythema in persons with darkly pigmented skin. A multispectral imaging approach was selected based upon its compatibility with the goal of developing a handheld erythema detection device to enhance visual assessment of the skin by clinicians.

The multispectral image acquisition system for this was based on a Dragonfly™ CCD camera (Point Grey Research, Inc,), which has a resolution 640×480 pixels with eight (8)-bit grey-scale per pixel. Twelve (12) optical filters with center wavelengths ranging from 400 nm to 950 nm at 50 nm intervals were mounted in a rotating motorized filter wheel. A pair of 40 W incandescent bulbs provided illumination. Multispectral images were acquired by sequentially changing filters in front of the camera lens, resulting in a series of images with a focused spectral region. The use of twelve filters covering the UV to near IR spectral range permitted analysis and identification of the most important spectra for erythema detection.

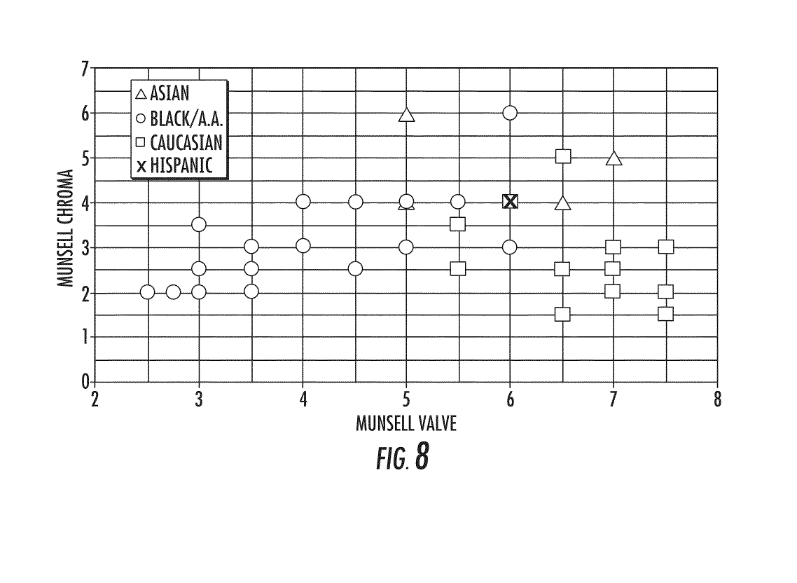

Erythema was mechanically induced by a pneumatic cuff and indenter on the shanks of 60 subjects. The medial tibial flare was selected as the loading site because it is relatively flat and is able to be loaded without discomfort. The erythema location was marked by four dots using black eyeliner. A color picture was taken of the test area with a Sony Mavica digital camera (Sony Corporation) for reference. Then, the multispectral images for the subject were taken by the prototype of the image acquisition system. For every subject, five (5) to nine (9) sets of images were taken with different gain and shutter settings in the first 150 seconds after pressure was released. Multiple gain settings were used to insure that an adequate response was received across all filters and skin tones. Data from 56 subjects was included in the analysis. Reasons for data exclusion included the image not capturing the entire region of erythema and instrumentation error. Self-reported race and ethnicity information was collected with the following breakdown: 28 black/African-American, 17 white/Caucasian, 5 Asian, 2 Hispanic and 4 were unreported. Skin color was recorded for each subject using a Munsell Color Chart with hue SYR. The measurements were given in terms of chroma and value, and were recorded for the closest matching sample on the soil color chart. FIG. 8 shows the distribution of the subjects' skin color; subjects are identified by race and ethnicity information.

Raw images were pre-processed before identifying a region of interest (ROI), defined as the region with erythema, and a region of uninvolved skin. Each image was cropped to a size that included only the subject's leg. The image set consisted of 12 separate pictures, requiring registration before analysis. Image registration is the process of aligning the different images into one coordinate system and is necessary to combine or fuse images into one picture. The cropped images were registered to a base image, which was the 550 nm image.

Cross-correlation values were used to register the images, using a method similar to those described by Bourke and Brown. For each image, I, a cross-correlation series was generated which contained correlation coefficients between the base image and I (x,y), where x and y are the amount that I is shifted. In most cases, the <x,y> pair that produced the greatest correlation coefficient was the shift needed to register the image; however, this was not always the case. To reduce the occurrence of producing a wrongful shift, a value p was added to the correlation coefficient. The value p is equal to the prior probability of the shift being <x,y>, multiplied by an empirically determined constant k. The prior probability of <x,y> was calculated from 2-D Gaussian curves that were estimated from the distribution of observed registration error in a randomly chosen subset of the images. The <x,y> pair that produced the maximum summed value of the correlation coefficient at <x,y> and p(x,y), was taken as the shift needed to register the image. This method was observed to reduce the occurrence of shifting to a less accurate position, over just using correlation values.

Shading correction was done by fitting a paraboloid to each image using ‘lsqcurvefit’, a least squares curve fitting function provided by MATLAB (The MathWorks, Inc.). The generic equation of a paraboloid,

Z(x,y)=x²+y²+c (1)

was modified to add terms for x and y translation, scaling along the original axes, and rotation about the z axis. The resulting equation was

Z(x,y)=(a₁ cos(a₃)²+a₂ sin(a₃)²)*(x−a₄)²+(a₁ sin(a₃)²+a₂ cos(a₃)²)*(y−a₅)²+2((a₁−a₂)cos(a₃)sin(a₃))(x−a₄)(y−a₅)+a₆ (2)

After the best fit values for coefficients aₙ were calculated, the best fit curve C_{best} was known. For all points (x, y) ε the erythema image I, a difference value D(x, y) was calculated using Equation (3).

D(x,y)=max(C_{best})−C_{best}(x,y) (3)

The values of D were then added to the values of Ito get a shade corrected image.

The ROI was then located using the four marks that circumscribed the area of tissue to which the ischemic load was applied. The pixels comprising the ROI were hand selected from the central area defined by these marks. Three (3) areas of uninvolved skin were also hand selected from the areas surrounding the defined ROI. The spectral responses of these areas were averaged resulting in average spectral responses for erythematic and non-erythematic skin for each filtered image of every subject.

Multiple image fusion algorithms were tested to determine the abilities of each in detecting erythema. Several erythema detection algorithms have been reported in the literature, but only three were compatible with the multi-spectral approach used in this study; those described by Tronnier, Diffey, and Dawson. These were slightly modified to synch with the filters used in the study. In addition, two algorithms were developed within this study. The five (5) fusion algorithms used in this study were:

1. Dawson. Dawson based his algorithm on the area below the melanin absorption curve when an artificial baseline is drawn from 510 nm to 610 nm Because the multispectral system used in this study differed in spectral resolution and filter bandwidth from that used by Dawson, the resulting equation was:

E_{Daw}=100[4A₅₅₀−2(A₅₀₀+A₆₀₀)], where A=absorption at the noted wavelength (4)

Dawson applied a melanin compensation algorithm based on the differences between spectral information from the erythema chromophore (645 nm, 650 nm, and 655 nm) and those due mainly from melanin (695 nm, 700 nm, and 705 nm). The adapted equation for this study was:

M_{Daw}=100(A₅₅₀₋₆₀₀−A₆₅₀₋₇₀₀) (5)

The melanin compensation was then applied using the following formula (where γ=0.04):

E_{corrected}=E_{Daw}(1−γM_{Daw}) (6)

2. Diffey. Diffey based his algorithm on the premise that differences in red (635 nm) and green (565 nm) absorption reflected the Hb content in tissue. The equation, adapted to the multispectral system used in this study, was:

$${E}_{diffey} = {log}_{10} ( \frac{{REF}_{650}}{{REF}_{550}} ) , \\ where \\ REF = reflectance at the noted wavelength \qquad ( 7 )$$

3. Tronnier: Tronnier's erythema index considers the relative differences between red (661 nm) and green (545 nm) reflectance at control and erythematic sites. Because the algorithm was to be used as a fusion algorithm where pixel by pixel calculations were to be made, relative values were not calculated during fusion. The relative differences of the ROI and non-ROI in the fused images were left up to post fusion algorithms to interpret. The adapted equation used in this study was:

E_{tronnier-used}=(REF₅₅₀−REF₆₅₀) (8)

4. GT-A algorithm: Tissue Chromophores. An algorithm was developed based upon the known chromophores of erythematic and non-erythematic skin. The oxy- and deoxy-hemoglobin absorption peaks were represented by the 550 nm filter. The 950 nm filter was used to capture the water peak between 940 nm and 970 nm, which helped improve contrast because of the increased water content in erythematic skin. The 650 nm filter was used to reflect melanin content, which was constant across erythematic and non-erythematic skin The resulting algorithm used in this study was:

I_{GT-A}=(2*REF₅₅₀−REF₆₅₀)*REF₉₅₀ (9)

5. GT-B algorithm: Filter optimization approach. Optimal Index Factor (OIF) is a statistical analysis algorithm based on the variance and correlation of filter bands that is used to determine the most informative filters. This function is mathematically described in Equation (10), where σ₁ is the standard deviation of band i, rᵢⱼ is the correlation coefficient between the filter band i and j image, and n is the number of multispectral images. If images contain non-uniform information and the relationships between them are weak, the OIF value will be high.

$$OIF = ( {\sum }_{i}^{n} {σ}_{i} ) \text{/} ( {\sum }_{j}^{n} \vert {r}_{ij} \vert ) \qquad ( 10 )$$

The OIF algorithm was run against all combinations of filters for each subject. The output of routine reported the specific filters that contained complex and unique information for each subject. The top three filters from each subject were tabulated for all subjects. The four filters that were listed most often were: 450 nm, 500 nm, 550 nm, and 950 nm.

Randomized Hill Climbing (RHC) was then used to search through a subspace of fusion algorithms that used the four (4) filters. The subspace consisted of all fusion algorithms in the form of:

$${I}_{Fused} ( a ) = \frac{{a}_{1}^{*} {REF}_{450} + {a}_{2}^{*} {REF}_{500} + {a}_{3}^{*} {REF}_{550} + {a}_{4}^{*} {REF}_{950}}{{a}_{5}^{*} {REF}_{450} + {a}_{6}^{*} {REF}_{500} + {a}_{7}^{*} {REF}_{550} + {a}_{8}^{*} {REF}_{950}} , a ∈ {ℜ}^{8} , - 1 \leq {a}_{i} \leq 1 \qquad ( 11 )$$

This particular set of fusion algorithms consists of combinations of the four filters such that the numerator is a linear combination of the four REF images and the denominator is a linear combination of the four REF images. After the fused image is created, it is normalized to the range [0, 255] so it can be evaluated.

The value function for the RHC algorithm, as shown in Equation (12), reflects the mean difference between the ROI and non-ROI of the fused images for a group of subjects.

$$val ( a ) = \frac{{\sum }_{s ∈ S}^{ } {μ}_{ROI} ( {I}_{Fused} ( a ) ) - {μ}_{non - ROI} ( {I}_{Fused} ( a ) )}{\vert S \vert} \qquad ( 12 )$$

The RHC algorithm was applied to two sets of subjects, the set of all subjects (consisting of four (4) ethnicities) and the set of only black subjects. Both sets produced good algorithms, but in order to better address the purpose of this research only the results from the black-only set will be discussed. Below the fusion algorithm is shown with the coefficients filled in.

$${I}_{GT - B} = \frac{\begin{matrix}- 0.56 * {REF}_{450} + 0.33 * {REF}_{500} + \\ 0.28 * {REF}_{550} + - 0.32 * {REF}_{950}\end{matrix}}{\begin{matrix}0.66 * {REF}_{450} + - 0.3 * {REF}_{500} + \\ - 0.13 * {REF}_{550} + 0.57 * {REF}_{950}\end{matrix}} \qquad ( 13 )$$

A general definition of image fusion is given as the combination of two or more different images to form a new image by using a certain algorithm. The algorithms defined above were used to create fused images. In addition, an image histogram equalization algorithm was applied to improve the image contrast and erythema visibility. The histogram equalization is a process of adjusting the image so that each intensity level contains an equal number of pixels. Therefore, each fusion algorithm was tested in two states, with and without histogram equalization.

The entire set of images, involving 5 erythema-enhancement algorithms and histogram equalization, were tested using two approaches. Each algorithm was initially evaluated using Weber contrast, a simple metric based upon the foreground (ROI) and background (non-erythematic skin) luminance (Equation 14). Work by Campbell suggests that human ability to perceive detail is affected by contrast and size of the image.

I−I_{B}/I_{B}, where I=ROI luminance & I_{B}=background luminance (14)

Two areas of each image background were identified as non-erythema. These two areas were combined with the ROI, or erythema region, resulting in three (3) areas used in detection accuracy. The use of non-erythema areas in detection analysis permitted assessment of how non-erythema sites are classified. Weber contrast values were calculated as if the selected area (erythema and two non-erythema) was the foreground (I). A simple threshold-based classification routine, using the J48 decision tree algorithm implementation in Weka, classified each data point. This routine reports detection accuracy and the classifications of each site. The true-positive, true-negative, false-positive and false-negative classifications of erythema were then used to calculate the sensitivity and specificity.

### EXAMPLE 2

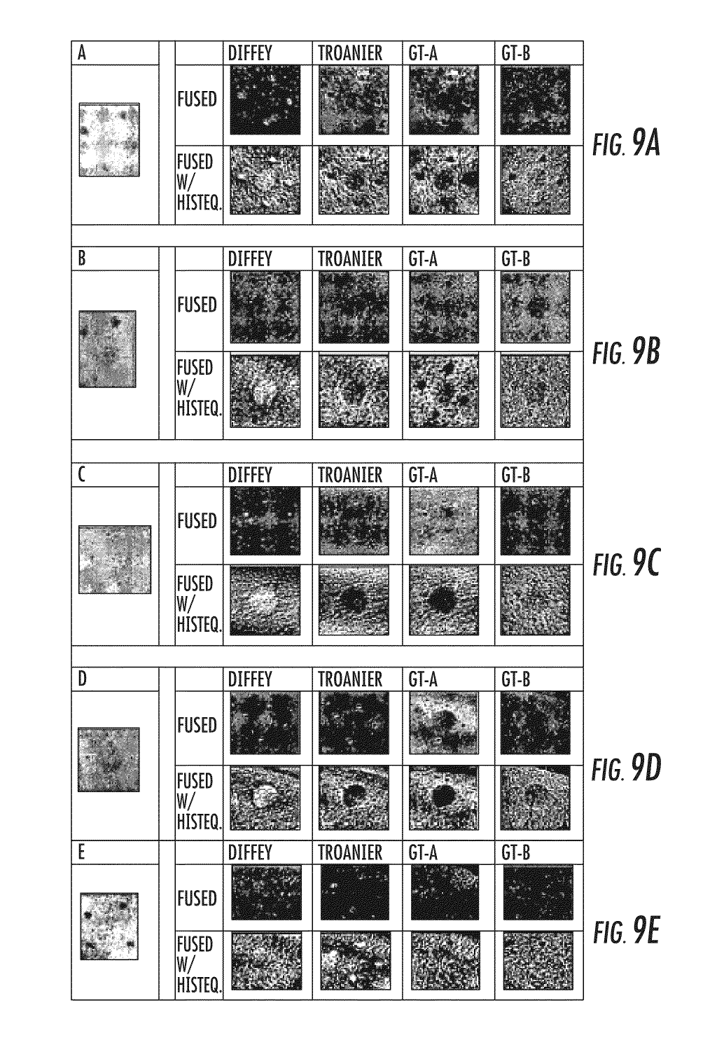

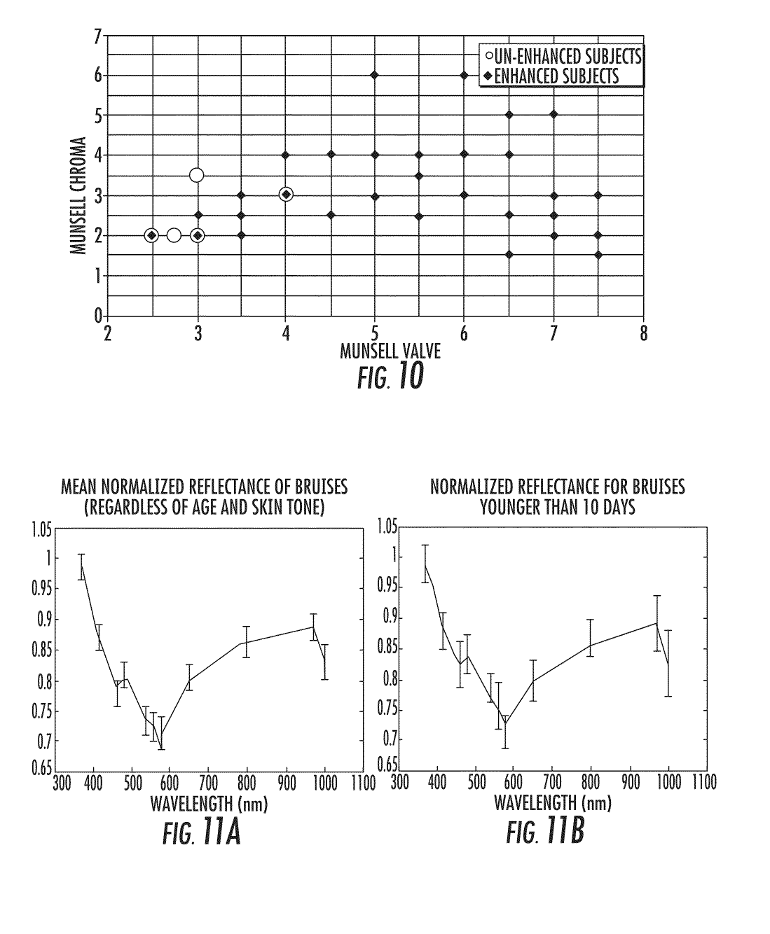

Ten (10) fused and enhanced images were created for each subject; one for each fusion algorithm and one for each of the fused images after histogram equalization. Fused images were visually compared with a digital camera image. The erythema contrast of the fused and enhanced images differed across algorithms and subjects. This could be due to many factors such as melanin content, image quality, etc. FIGS. 9A-E illustrates the fusion results of five representative subjects based on the four most effective algorithms.

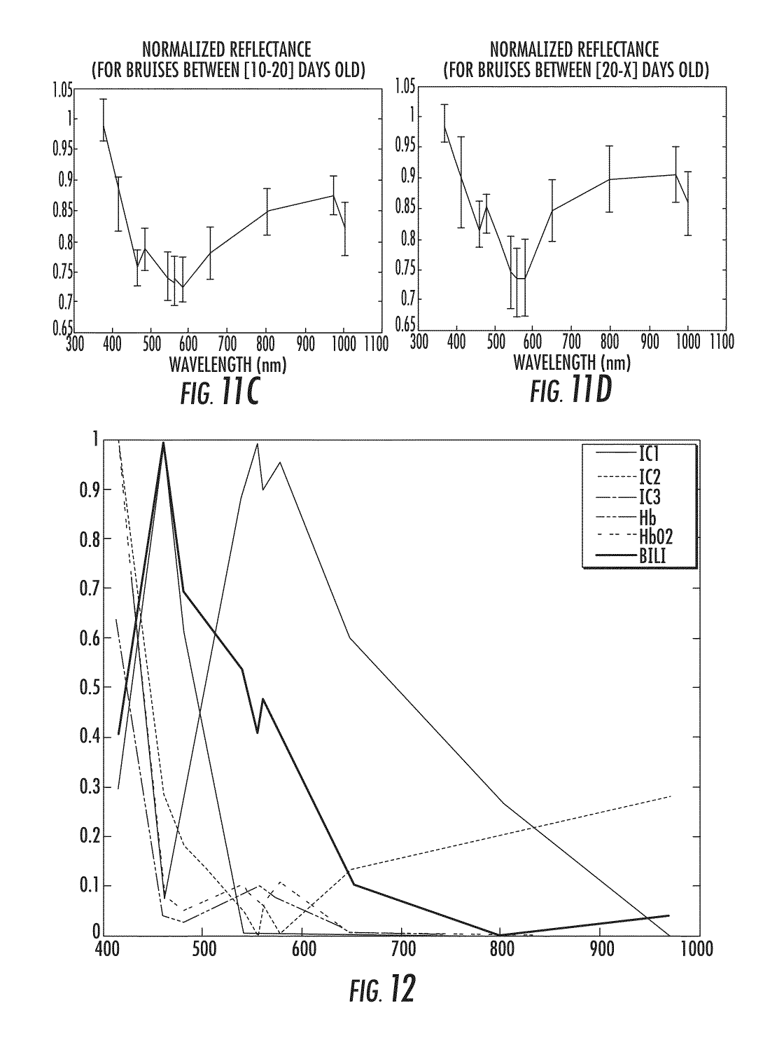

The Diffey, Tronnier, GT-A, and GT-B algorithms adequately enhanced erythema. Images from Subjects A and B illustrate the ability to create images where the erythema is discernable compared to the full spectral image taken by the digital camera. In subjects whose erythema is visible in a full spectral image (Subjects C and D), the fused images drastically enhance the erythema. All such enhancements could prove to be valuable in a clinician's assessment of erythema. The dataset also included induced erythema for which the fusion algorithms did not appreciably improve visualization (e.g. Subject E). The six subjects that fell into this group were among those with the darkest skin (see FIG. 10). This however, was not the fusion outcome for all subjects with similar levels of skin pigmentation, which suggests that other factors may contribute to the difficulty of enhancing erythema.

Although the visual comparison gives an overall subjective assessment of the fusion algorithms, Weber contrast was used for quantitative evaluation. The average Weber contrast value for the digital camera and fused images for each subject are shown in Table 1. Data from subjects who identified themselves as Black or African-American are shown as a subset of the entire dataset.

TABLE 1
Weber Contrast Values

| | Digital | Dawson | | Diffey | | Tronnier | | GT-A | | GT-B | |
|---|---|---|---|---|---|---|---|---|---|---|---|
| | Camera | no eq. | eq. | no eq. | eq. | no eq. | eq. | no eq. | eq. | no eq. | eq. |
| All Subj. | 0.031 | 0.030 | 0.131 | 0.138 | 0.568 | 0.158 | 0.531 | 0.201 | 0.628 | 0.088 | 0.280 |
| Black Subj. | 0.028 | 0.049 | 0.096 | 0.120 | 0.472 | 0.148 | 0.480 | 0.182 | 0.544 | 0.078 | 0.226 |
| only | | | | | | | | | | | |

Average contrast values increased from the digital camera images to the fused images for each fusion algorithm except for the Dawson algorithm. Concentrating on the Black subject subset, Diffey, Tronnier and GT-A resulted in a four (4)-fold contrast increase. With histogram equalization, Diffey, Tronnier and GT-A algorithms had a 17× increase in contrast.

Each datapoint was classified as erythema or non-erythema. The dataset consisted of ⅓ erythema sites since two non-erythema or control sites were also analyzed for each person. The value of correctly classified instances represents the percentage of times that the classifier correctly identified the site, over both sets of data. Sensitivity is the proportion of true positives that were correctly identified whereas specificity is the proportion of true negatives identified by the algorithm. Sensitivity, specificity, and percentage of correctly classified instances are shown for each fusion algorithm, with and without histogram equalization (Table 2). Table 3 contains similar information for the set of only black subjects. For the Black subject subset, the Diffey and Tronnier algorithms with histogram equalization had at least 80% accuracy. The accuracy of the GT-A algorithm, with and without equalization, exceeded 90%. The Sensitivity of the GT-A algorithm was 1.0 meaning that all erythema sites were identified as having erythema.

TABLE 2
Accuracy and Predictive Values for All Subjects

| | | | Correctly Classified |
|---|---|---|---|
| Fusion Algorithm | Sensitivity | Specificity | Instances (%) |
| Dawson | 0 | .667 | 66.7 |
| With equalization | 0 | .667 | 66.7 |
| Diffey | .681 | .802 | 76.8 |
| With equalization | .619 | .838 | 75.6 |
| Tronnier | .679 | .839 | 78.6 |
| With equalization | .756 | .821 | 80.3 |
| GT-A | .778 | .933 | 87.5 |
| With equalization | .962 | .948 | 95.2 |
| GT-B | .667 | .767 | 74.0 |
| With equalization | .882 | .806 | 82.1 |

TABLE 3
Accuracy and Predictive Values for only Black Subjects

| | | | Correctly Classified |
|---|---|---|---|
| Fusion Algorithm | Sensitivity | Specificity | Instances (%) |
| Dawson | 0 | .667 | 66.7 |
| With equalization | 0 | .667 | 66.7 |
| Diffey | .581 | .881 | 72.6 |
| With equalization | .842 | .815 | 82.1 |
| Tronnier | .667 | .852 | 78.6 |
| With equalization | .895 | .831 | 84.5 |
| GT-A | 1 | .889 | 91.7 |
| With equalization | 1 | .918 | 94.0 |
| GT-B | .536 | .768 | 69.0 |
| With equalization | .833 | .75 | 76.2 |

The three most successful algorithms, Diffey, Tronnier, and GT-A, used two, two and three spectra, respectively. This result is consistent with the objective to identify a finite number of spectra that can be incorporated into an affordable, handheld clinical device.

The increased contrast values suggest that the fused images are more readable and the information encoded should be more discernable. This result corroborates the results of the subjective visual comparison of the images which found that Diffey, Tronnier, GT-A, and GT-B algorithms enhanced erythema detection in the majority of subjects. Moreover, the accuracy and discrimination values demonstrated the degree of class separation (erythema versus non-erythema) generated by the fusion algorithms.

### EXAMPLE 3

Sixteen residents of an extended care facility, who presented with bruising were recruited for the study. Ages ranged from 72-86 and the group consisted of 5 males and 2 African-American subjects. The multispectral image acquisition system described above was used to collect data. Images were taken between 2 and 9 times over the course of bruise resolution. A surgical marking pen was used to circumscribe the bruise to permit repeated imaging of the site and to define bruised skin from ‘normal’ skin during analysis.

Preprocessing included three sequential steps. First, the image was cropped to only include areas of skin around the bruise. Bruise sets with spatially shifted images were excluded from the study. Second, a shading correction procedure was used to reduce the pixel intensity variation caused by curvature of the skin. Third, a variable digital gain was applied to adjust the intensity range of the shade-corrected bruise image for the purpose of visual comparison.

Normalized bruise reflectance (NBR) was used to eliminate the effects of the unknown incidence light density. The NBR is defined as the optical reflectance coefficient of bruises over the optical reflectance coefficient of normal skin. NBR values were segmented by bruise age, location and other factors and graphed. In addition, visual contrast of the bruises were investigated using different fusion algorithms based upon literature review and related work. The approach selected decoupled the fusion of bilirubin from that of hemoglobin as a means to highlight both chromophores.

The bilirubin peak near 460 nm lies near a steep downward slope in the hemoglobin curves. After falling, the hemoglobin response rises again. At approximately 540 nm, the Hb response is about equal to that at 460 nm (the bilirubin peak). Our fusion algorithms contrast these points as a means to cancel the Hb response. In addition, melanin varies slowly over this 80 nm range, nearly canceling. The result is a contrast fusion algorithm with a high contrast bilirubin characteristic, without much contamination from other products. Hb was captured using a similar approach.

The 577 nm band corresponds to the upper hump of the well-known “W” of hemoglobin reflectance. The 650 nm frequency was selected as a contrast reference. As in the case of the bilirubin algorithm, the most salient difference in the absorption curves is the hemoglobin absorption, with all others contributing nearly equal absorptions at the two selected wavelengths.

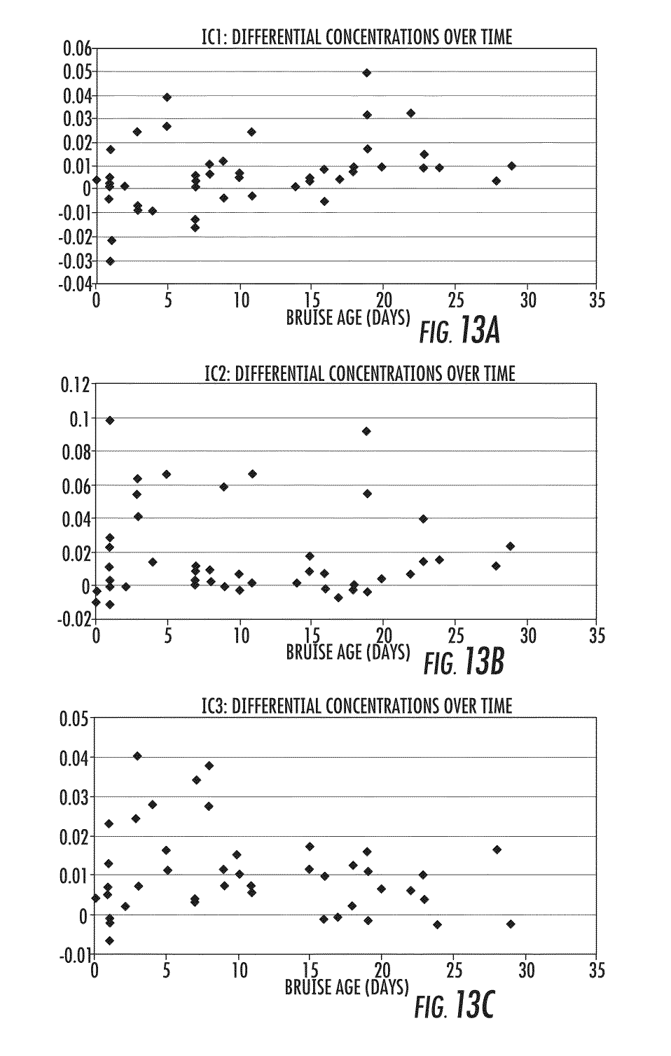

The mean NBR of all bruises were calculated and plotted in FIG. 11A against the center wavelength of the filters. The deep valley between 555-577 nm indicates high contrast between the bruise and normal skin around these wavelengths. This may suggest an accumulated pool of extravagated from the damaged capillaries of the bruised tissue since hemoglobin has absorption peaks in this region

Mean NBR for three bruise age groups <10 days, 11-20 days and >=21 days were plotted against the wavelength in FIGS. 11B, C & D respectively. All NBR curves have a common valley between 555 nm and 577 nm regardless of bruise age. This suggests that the pooled blood lingers in the bruised region quite a long time before disappearing. In addition, the NBR curve of the youngest bruises (FIG. 11B) is lower than the curves of the older bruises. This is a reasonable result since, as the bruise heals, its spectral response should approach that of normal skin. However, as indicated by the wide error bars associated with the NBR values, bruises exhibit tremendous variation, as no single NBR value can be used to derive bruise aging information.

Bilirubin has peak absorption at 460 nm NBR at 460 nm decreases from the <10 day bruise set (FIG. 11B) to the 11-20 day bruise set (FIG. 11B) before rising again. This drop of NBR at 460 nm is consistent with Randeberg, who observed that bilirubin concentration is higher during the first lo days after bruising before decreasing. However, despite this common trend, a high variability across bruises is evident. Potential influences include the anatomical location, the difference in underlying tissue, and the cause of a bruise (impact versus a needle stick).

Bruises show a strong contrast against normal skin at wavelengths centered between 555 nm and 577 nm, corresponding to the hemoglobin response. This contrast remains as bruises age. Bruises in elderly people show a wide variation of spectral response over time and a wide timeframe for resolution. Some bruises in the study did not resolve after 45 days. A relatively simple, yet consistent, metric for bruise aging features in multi-spectral images is proposed. If shown to be robust, the development of an inexpensive, clinically-viable bruise detection device is possible.

### EXAMPLE 4

A commonality between the existing methods of bruise aging is analysis of bruise color or estimation of chromophore concentration. Changes in bruise color are attributed to the breakdown of hemoglobin. The breakdown of hemoglobin causes the relative chromophore concentration to change, which is responsible for spectral response that determines observed bruise color. In this study, a method of chromophore concentration estimation that can be employed in a handheld imaging spectrometer with a small number of wavelengths was investigated. The method is capable of giving differential concentration estimates without measuring incident light. Using the method to build a model of differential concentration estimates and known bruise age, we hope to be able to estimate the age of a bruise in question.

Residents of an extended care facility and students and staff of our lab were recruited for the study. Ages ranged from 19-86. Subjects were divided into two groups consisting of subjects younger than 65 years of age and subjects at least 65 years of age. Subjects of the former group had much more consistent bruising patterns and time for resolution than did the subjects of the latter group. Only subjects of the former group, younger than 65 years of age are analyzed in this study.

The multispectral image acquisition system was based on a Unibrain Fire-i™ CCD camera (Unibrain, S.A.), having a resolution of 640×480 pixels and eight (8)-bit grey-scale per pixel. The rotating motorized filter wheel could accommodate 12 filters. The system was fit with 11 bandpass filters (FWHM: 10 nm-40 nm) with center wavelengths targeting the absorption peaks of the primary chromophores of blood and skin. Multispectral images were acquired by sequentially changing filters in front of the camera lens, resulting in a series of images with a discrete spectral region. A rectangular array of four (4) 40 W halogen bulbs provided illumination. The system was controlled using Labview.

Multispectral images were preprocessed before analysis. The first step was cropping the image to include only areas of the subject's skin. The second step was correction of shading due to surface curvature. This was done by fitting a quadratic surface to the areas of skin that did not consist of bruised skin. The image was then corrected by subtracting the offset of the surface from mean value of the surface at each coordinate.

ROI selection was also included in the preprocessing stage. A rectangular region, B, of pixels in the bruised skin was selected as the Bruise ROI. Four (4) rectangular regions of adjacent normal skin, N₁₋₄, were selected as the Normal Skin ROI. Because values were averaged over B and N, the native registration accuracy of the multispectral image sufficed allowing us to omit any further registration.

Normalized bruise reflectance (NBR) values were calculated by dividing the mean pixel value of B by the mean pixel value of N. According to Beer-Lambert's law, the negative log of the calculated NBR is equal to the absorbance difference, A_{d}, between the bruised skin and normal skin (eq. 15).

A_{d}=A_{b}−Aₙ=−log(NBR) (15)

The Beer-Lambert law asserts that total absorbance is the sum of absorbance from each of the constituent absorbers (eq. 16). Absorbance due to each absorber is the product of the absorption coefficient α of the absorber, the path length (l) the light travels through the absorber, and the concentration (c) of the absorber.

A=Σᵢαᵢlᵢcᵢ (16)

If a constant path length between normal skin and bruised skin is assumed, A_{d} can be put in terms of the concentration difference of each absorber (eq. 17).

A_{d}=A_{b}−Aₙ=Σᵢαᵢlᵢ(c_{ib}−cᵢₙ)=Σᵢαᵢlᵢc_{id} (17)

Then, a system of equations X=MS can be set up, where X contains the A_{d} values for each λ for each multispectral image in our data set, our observed data. Columns of X correspond to λ₁ through λ₁₁. Rows of X correspond to different multispectral images, each of which represents a bruise on a subject at a certain age of the bruise. Some subjects had multiple bruises imaged and most bruises were imaged on several different days.

$$X = [ \begin{matrix}{A}_{d} ( {λ}_{1} , 1 ) & … & {A}_{d} ( {λ}_{11} , 1 ) \\ ⋮ & ⋱ & ⋮ \\ {A}_{d} ( {λ}_{1} , m ) & … & {A}_{d} ( {λ}_{11} , m )\end{matrix} ] \qquad ( 18 )$$

S contains the values of the absorption coefficients for each absorber at each wavelength, our source signals. Again, columns of S correspond to λ₁ through λ₁₁. Rows of S correspond to the different absorbers ss₁ to ssₙ.

$$S = [ \begin{matrix}α ( {λ}_{1} , {ss}_{1} ) & … & α ( {λ}_{k} , {ss}_{1} ) \\ ⋮ & ⋱ & ⋮ \\ α ( {λ}_{1} , {ss}_{n} ) & … & α ( {λ}_{k} , {ss}_{n} )\end{matrix} ] \qquad ( 19 )$$

M is the mixing matrix, and demonstrates how to mix the different source signals to get X. Under the assumption of constant path length, M represents concentration difference of absorbing constituents in bruised relative to normal skin.

S can be generated in two different ways. The first way is by independent component analysis (ICA), using a method similar to Polder. ICA is a form of source signal separation that estimates source signals and the mixing matrix given the observed signals. In general, the discovered independent components can be interpreted as underlying causes of observations, especially when one believes that: (1) observed features are generated by the interaction of a set of independent hidden random variables, and (2) these hidden variables are likely to be kurtotic (i.e. each variable is discriminative and sparse). ICA has-been used in a variety of blind source separation problems including audio source separation, image segmentation, and fMRI of the brain. In this work, a highly efficient version of the ICA algorithm, FastICA, was used.

The other method of generating S is by using the published absorption coefficient data from literature. Using this method, S was generated with absorption coefficient data for deoxygenated-hemoglobin (Hb), oxygenated-hemoglobin (oxyHb), and bilirubin. S was also generated with absorption coefficient data for just Hb and bilirubin. Work by Randeberg suggests that Hb and bilirubin are the two chromophores that are most important in bruise aging.

Estimation of M is done differently, depending on how S is generated. When FastICA generates S(S—ICA), M is generated simultaneously. When using published values of S(S-Lit), M is generated by finding the least-squares fit in MATLAB (The Mathworks, Inc.).

Before source signals were estimated with ICA, dimensionality reduction was done with PCA. We reduced the data to 3 dimensions and then estimated 3 sources to fit a model where the absorbers were Hb, oxyHb, and bilirubin. The resulting ICs are plotted with the published absorption coefficient values in FIG. 12. Estimates of the differential concentration and path length given in the corresponding mixing matrix M are plotted in FIGS. 13A-C. This figure contains data for all of the subjects.

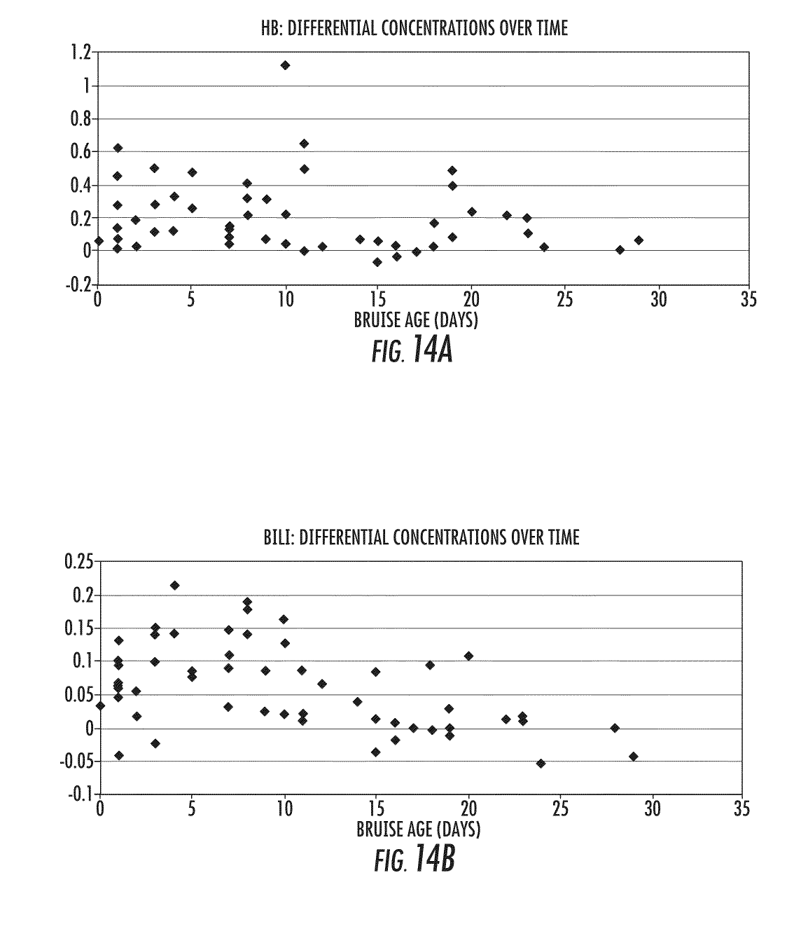

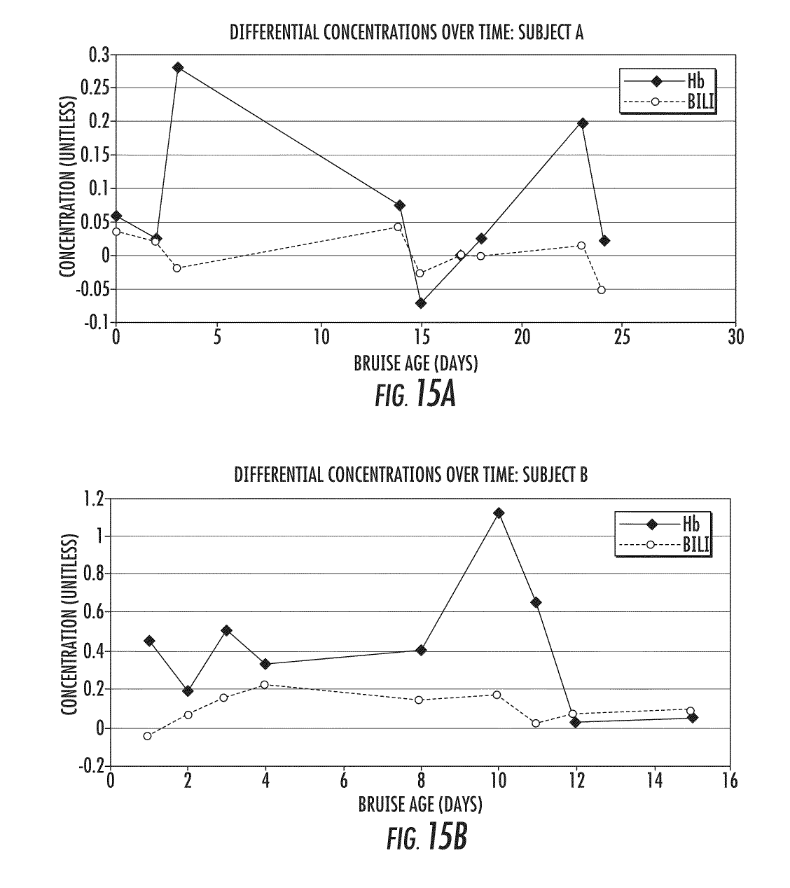

Estimation of M using published sources was done for two different S's. S-Lit₁ was composed of absorption coefficients for Hb, oxyHb, and bilirubin. S-Lit₂ was composed of values for Hb and bilirubin. The differential concentration and path length values for all subjects, from M, are plotted versus bruise age for S-Lit₂ in FIGS. 14A-B. FIGS. 15A-B contain this data plotted for two subjects, Subject A and Subject B. Each subject was 38+/−2 yrs, Caucasian, and the imaged bruise was on the thigh.

The differential concentration estimates for all subjects (FIG. 14) show high variation, but they also show general trends. Day one (1) (24-48 hours after the trauma event) was the first day the majority of the bruises were imaged. According to Langlois, bleeding may continue for 24-48 hours after the trauma event. This would result in a maximal concentration of Hb and oxyHb in that period. Breakdown of Hb would occur slowly over a matter of days. The trend seen in differential Hb concentration (FIG. 14) is decreasing concentration with time. Bilirubin is a breakdown product of Hb and begins to form several hours after the trauma event. Yellow color, produced by bilirubin, has been reported to peak in the range of 6 to 12 days after trauma. The differential concentration of bilirubin in FIG. 14 appears to increase until it peaks between day four (4) and nine (9), and then it decreases with time.

Data for the two individual subjects does not obviously demonstrate the trends. For Subject A the plots seem random and values do not appear to have any correlation with time. On the other hand, the trends can be picked out of the data for Subject B. Bilirubin peaks out between four (4) and ten (10) days and tends to zero at the later days.

A technique for estimating differential concentrations of chromophores present in bruises has not previously been published. This above-described technique can be employed in a multispectral imaging system employing a small number of wavelengths. This study revealed that using a small number of wavelengths (11), one can estimate differential concentrations of chromophores in bruised skin. The employed model would allow use of as few wavelengths as there are chromophores. In addition, estimates of differential concentration and path length appear to be a good indicator of chromophore concentration versus time, as given by published models. Finally, the reliability of differential concentration estimate that can be given by a single image is questionable due to high variance. It is therefore seems reasonable to investigate a method of improving the estimation.

What is claimed is:

1. A method for non-invasive detection, identification or characterization of erythema or bruising of skin beyond visual inspection with the unaided eye, the method comprising the steps of:
transmitting light at a different wavelengths within a plurality of narrow spectral regions to the skin to illuminate the skin;
capturing a plurality of separate spectral images of the skin using a multi-spectral filter array directly mounted to a single sensor; and
processing the images to form a real time composite image of the skin to detect, identify or characterize erythema or bruising of skin.
